(19) United States
(12) Patent Application Publication
(10) Pub. No.: US 2009/0156467 A1
(43) Pub. Date: Jun. 18, 2009

(54) Thymic stromal lymphpoietin promoter and use therefor

(76) Inventors: Zhengbin Yao, Berwyn, PA (US); Guanghui Hu, Phoenixville, PA (US); Yucheng Li, San Diego, CA (US); Kang Li, San Diego, CA (US)

Correspondence Address: Nikolaos C. George;JONES DAY, 222 E. 41st. Street, New York, NY, 10017-6702, US

(21) Appl. No.: 11/658,414

(22) PCT Filed: Jul. 27, 2005
(86) PCT No.: PCT/US05/26633

§ 371 Date: Nov. 14, 2008

Related U.S. Application Data
(60) Provisional application No. 60/591,719, filed on Jul. 28, 2004.

(51) Int. Cl. A61K 38/02; C12N 15/11; C12N 15/00; C12N 5/08; C12Q 1/66; C12Q 1/02; C12Q 1/68; A01K 67/027; A61K 31/7088; A61K 31/20

(52) U.S. Cl. 514/8; 536/24.1; 435/320.1; 435/366; 435/8; 435/29; 435/6; 800/13; 514/2; 514/44; 514/558; 514/23

(57) ABSTRACT

A promoter comprising the isolated promoter region of the human Thymic Stromal Lymphopoietin (TSLP) gene and functional portions of the promoter region having TSLP promoter activity. The promoters are useful for identifying promoter agonists and antagonists that can be used to prevent or treat allergic conditions and autoimmune diseases.

### FIELD OF THE INVENTION

This invention relates generally to promoters and particularly to the promoter region of the human Thymic Stromal Lymphopoietin (TSLP) gene and its use to develop agents for the prevention and treatment of allergic conditions and autoimmune diseases.

### BACKGROUND OF THE INVENTION

TSLP belongs to a helical bundle type I family of cytokines. Other members of this family include interleukin-2 (IL-2), IL-4, IL-5, IL-9, IL-15 and IL-21. TSLP was originally identified from the conditioned medium of a mouse thymic stromal cell line that promoted the development of B-cells. Mouse TSLP shares approximately 43% amino acid sequence identity with human TSLP. Park et al., J. Exp. Med. 192:659-670 (2000); Quentmeier et al., Leukemia 15: 286-92 (2001); and U.S. Pat. No. 6,555,520.

TSLP has been found to mimic the activity of interleukin-7 (IL-7) by stimulating the production of pro-allergic cytokines from T_{H}2 cells. Human TSLP has also been found to potently activate CD11c+ dendritic cells (DC) and induce production of the T_{H}2 attracting chemokines Thymus and Activation-Regulated Chemokine (TARC) and Macrophage-Derived Chemokine (MDC). Gilliet et al., J. Exp. Med. 197:1059-63 (2003). TSLP-activated DCs can induce strong näive CD4+ T cell proliferation and prime T_{H}2 cells to produce IL-4, IL-5, IL-13 and tumor necrosis factor-α (TNF-α), while down-regulating IL-10 and interferon-γ (INF-γ).

Human TSLPs have been found to be expressed by epithelial cells, lung fibroblasts, lung smooth muscle cells, other stromal cells and mast cells that are activated by IgE cross-linking. TSLP is also highly expressed in the skin lesions of patients with acute and chronic atopic dermatitis and may be associated with Langerhans cell activation. Soumelis et al., Nature Immunol. 3:673-80 (2002). Exemplary nucleotide and amino acid sequences of human TSLP include GenBank™ Accession No. AF338732, AY037115, NM_{—}033035, NM_{—}138551, NP_{—}149024 and NP_{—}612561. The region of the human chromosome containing the human TSLP promoter was cloned as a part of the cloning of human chromosome 5 (GenBank™ Accession No. AC008572 and NT_{—}034772). However, the region has not been characterized as a TSLP promoter.

TSLP also mimics the activity of IL-7 in supporting B-cell development. TSLP promotes the proliferation and differentiation of committed B220⁺ B-cell progenitors (pro-B-cell stage of differentiation) from day 15 fetal liver. Furthermore, TSLP mimics the activity of IL-7 in supporting the progression of B-cells from uncommitted bipotential precursors. The progeny of cells that give rise to mature B-lymphocytes fail to develop from these bipotential precursors if either TSLP or IL-7 is absent. Levin et al., J. Immunol. 162: 677-683 (1999). Thymic stromal lymphopoietin (TSLP) has been identified as an important cytokine in immunological cascades and in B-cell development. These findings suggest a role for TSLP in supporting the development of cells that are integral to the immunological response to antigen presentation and are therefore involved in allergic reactions and autoimmune responses.

Allergic and autoimmune responses and diseases are a result of complex immunological cascades that are triggered by various stimuli and are manifested in different ways. For example, Type I hypersensitivity reactions are mediated by IgEs or helper T cells (T_{H}2) that activate mast cells and can lead to allergic rhinitis (hay fever), asthma or systemic anaphylaxis. Similarly, Type IV hypersensitivity reactions are mediated by T cells that activate macrophages and can lead to contact dermatitis. Allergic reactions are problematic for many individuals. Each year more than 50 million Americans are thought to suffer from some kind of allergic disease. American Academy of Allergy, Asthma and Immunology's The Allergy Report: Science Based Findings on the Diagnosis & Treatment of Allergic Disorders, 1996-2001. In addition to the physical toll to the individual, allergic reactions have economic consequences. Allergies are the sixth leading cause of chronic disease in the United States, costing the health care system about $18 billion annually. There is, therefore, a need for drugs for preventing and treating allergic conditions and autoimmune diseases.

### SUMMARY OF THE INVENTION

It is, therefore, an object of the invention to provide a promoter useful for developing methods and compositions for preventing or treating allergic conditions and autoimmune diseases.

It is another object of the invention to provide methods and compositions for preventing or treating allergic conditions and diseases.

It is a further object of the invention to provide methods and compositions for preventing or treating autoimmune diseases.

These and other objects are achieved by identifying the nucleic acid sequence of the TSLP promoter and using the promoter to identify TSLP promoter antagonists and TSLP agonists that can be used as agents for preventing and treating allergic and autoimmune diseases.

Other and further objects, features, and advantages of the present invention will be readily apparent to those skilled in the art.

### DETAILED DESCRIPTION OF THE INVENTION

### Definitions

For convenience, certain terms and phrases employed in the specification, examples, and appended claims are defined herein. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

“Allergy” or “allergic condition” means any disease or disorder resulting from an immunological cascade (including Type I and Type IV hypersensitivity reactions) and is typically triggered by stimuli. Examples of allergic conditions include: allergic rhinitis, hay fever, perennial rhinitis, seasonal/perennial allergic conjunctivitis, vernal keratoconjunctivitis, giant papillary conjunctivitis, perennial allergic conjunctivitis and atopic keratoconjunctivitis, atopic dermatitis, and allergic asthma, food reactions, systemic anaphylaxis, allergic pulmonary disease, anaphylaxis, urticaria and angioedema (hives; giant urticaria; angioneurotic edema), hereditary angioedema, mastocytosis, physical allergy to physical stimuli, e.g., cold, sunlight, heat; mild trauma, contact dermatitis, hypersensitivity pneumonitis, allograft rejection, granulomas due to intracellular organisms, drug sensitivity, thyroiditis, encephalomyelitis after rabies vaccination, cryoglobulinemia, cryoglobulinemic glomerulonephritis, histiocytic lymphomas, Severe Combined Immunodeficiency (SCID), and tonsillitis.

“Autoimmune diseases” means conditions characterized by a specific humoral or cell-mediated immune response against constituents of the body's own tissues, such as self-antigens or autoantigens. Examples of autoimmune diseases include but are not limited to Active Chronic Hepatitis, Addison's Disease, Anti-phospholipid Syndrome, Atopic Allergy, Autoimmune Atrophic Gastritis, Achlorhydra Autoimmune, Celiac Disease, Crohn's Disease, Cushing's Syndrome, Dermatomyositis, Diabetes (type 1), Discoid Lupus, Erythematosis, Goodpasture's Syndrome, Grave's Disease, Hashimoto's Thyroiditis, Idiopathic Adrenal Atrophy, Idiopathic Thrombocytopenia, Insulin-dependent Diabetes, Lambert-Eaton Syndrome, Lupoid Hepatitis, some cases of Lymphopenia, Mixed Connective Tissue Disease, Multiple Sclerosis, Pemphigoid, Pemphigus Vulgaris, Pernicious Anema, Phacogenic Uveitis, Polyarteritis Nodosa, Polyglandular Auto. Syndromes, Primary Biliary Cirrhosis, Primary Sclerosing Cholangitis, Psoriasis, Raynaud's Syndrome, Reiter's Syndrome, Relapsing Polychondritis, Rheumatoid Arthritis, Schmidt's Syndrome, Limited Scleroderma (or CREST Syndrome), Severe Combined Immunodeficiency Syndrome (SCID), Sjogren's Syndrome, Sympathetic Ophthalmia, Systemic Lupus Erythematosis, Takayasu's Arteritis, Temporal Arteritis, Thyrotoxicosis, Type B Insulin Resistance, Ulcerative Colitis, and Wegener's Granulomatosis.

“TSLP agonist” means a compound that upregulates (e.g. potentates or supplements) the transcriptional activity at the TSLP promoter resulting in increased transcription of TSLP or a gene operably linked to the TSLP promoter. A TSLP agonist can be a compound that promotes the interaction of a transcription factor with a portion of the TSLP promoter (i.e., by protein-DNA interactions). Alternatively, a TSLP agonist may affect a transcription factor such that the transcription factor interacts with other transcription factors or components of the transcriptional machinery (i.e., by protein-protein interactions).

“TSLP antagonist” means a compound that downregulates (e.g. potentates or supplements) the transcriptional activity at the TSLP promoter resulting in decreased transcription of TSLP or a gene operably linked to the TSLP promoter. A TSLP antagonist can be a compound that inhibits or reduces the interaction of a transcription factor with a portion of the TSLP promoter (i.e., by protein-DNA interactions). Alternatively, a TSLP antagonist may affect a transcription factor such that the transcription factor does not interact with other transcription factors or components of the transcriptional machinery (i.e., by protein-protein interactions).

“Carbohydrates” means organic compounds that consist of carbon, hydrogen and oxygen. Carbohydrates vary from simple sugars containing from three to seven carbon atoms to very complex polymers. Classification of carbohydrates relates to their structural core of simple sugars, saccharides. Principal monosaccharides are glucose and fructose. Three common disaccharides are sucrose, maltose and lactose. Polysaccharides include, for example, starch, dextrin, glycogen and cellulose.

“Cells,” “host cells” or “recombinant host cells” are terms used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

“Homology” or “identity” or “similarity” means sequence similarity between two nucleic acid molecules. Homology can be determined by comparing a position in each sequence that may be aligned for purposes of comparison. When a position in the compared sequence is occupied by the same base, then the molecules are identical at that position. A degree of homology or similarity or identity between nucleic acid sequences is a function of the number of identical or matching nucleotides at positions shared by the nucleic acid sequences. An “unrelated” or “non-homologous” sequence shares less than 40% identity, though preferably less than 25% identity, with the TSLP promoter or portions thereof.

“Homogenate” means material that has been homogenized, especially tissue that has been ground and mixed. Preferred embodiments of homogenates include those from cells that have been transformed with a reporter gene operably linked to a TSLP promoter or portion thereof. Homogenates may also refer to cellular lysates. Homogenates may be partially purified or purified such that DNA, polypeptides, lipids and carbohydrates are partially or entirely removed from the homogenate.

“Interact” means detectable interactions (e.g. biochemical interactions) between molecules, such as interaction between protein-protein, protein-nucleic acid, nucleic acid-nucleic acid, and protein-small molecule or nucleic acid-small molecule in nature.

“Isolated” means nucleic acids, such as DNA or RNA, which are in a relatively purified state, i.e. separated from molecules that associate with the nucleic acid. For example, an isolated nucleic acid containing the TSLP promoter preferably includes no more than 5 kilobases (kb) of nucleic acid sequence that naturally immediately flanks the TSLP gene in genomic DNA, and more preferably no more than 3 kb of such naturally occurring flanking sequences. The term isolated as used herein also means a nucleic acid that is substantially free of cellular material, viral material, or culture medium when produced by recombinant DNA techniques, or chemical precursors or other chemicals when chemically synthesized. Moreover, an “isolated nucleic acid” is meant to include nucleic acid fragments that are not naturally occurring as fragments and would not be found in the natural state. Methods for isolating DNA are well known in the art. See, for example, Sambrook et al., Molecular Cloning: A Laboratory Manual, 2nd ed., Cold Spring Harbor Laboratory, Cold Spring Harbor, N.Y. (1989)).

“Lipids” means fatty acid esters, a class of relatively water-insoluble organic molecules, which are the “basic” components of biological membranes. Lipids may be found as phospholipids, steroids and triglycerides. Lipids consist of a polar or hydrophilic (attracted to water) head and one to three nonpolar or hydrophobic (repelled by water) tails. The hydrophobic tail consists of one or two (in triglycerides, three) fatty acids. Lipids may comprise unbranched chains of carbon atoms, which are connected by single bonds alone (saturated fatty acids) or by both single and double bonds (unsaturated fatty acids). The carbon chains of lipids are usually 14-24 carbon groups long.

“Nucleic acid” means polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides.

“Operably linked” means a nucleic acid linked to a TSLP promoter or portion thereof in a manner that allows expression of the nucleotide sequence. In a preferred embodiment, the nucleotide sequence is a reporter gene.

“Polypeptide,” “peptide” and “protein” are used interchangeably herein to mean polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” means either natural and/or unnatural or synthetic amino acids, including glycine and both the D or L optical isomers, and amino acid analogs and peptidomimetics.

“Portion” or “portions” means fragments of the TSLP promoter that function as DNA interaction sites for transcription factors and therefore have TSLP promoter activity, particularly those sequences given in Table 1.

“Reporter gene” means a gene whose phenotypic expression is easy to monitor. Preferred reporter genes encode enzymes, such as luciferase.

“Small molecule” means a composition, which has a molecular weight of less than about 5 kD and most preferably less than about 4 kD. Small molecules can be nucleic acids, peptides, polypeptides, peptidomimetics, carbohydrates, lipids or other organic (carbon containing) or inorganic molecules. Libraries of chemical and/or biological mixtures, often fungal, bacterial, or algal extracts, which can be screened with any of the assays of the invention to identify compounds that modulate TSLP promoter activity.

“Transgene” means a nucleic acid of the TSLP promoter or portion thereof that is operably linked to a reporter gene, the construct of which has been introduced into a cell. A transgene could be partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location that differs from that of the natural gene or its insertion results in a knockout). A transgene can also be present in a cell in the form of an episome. A transgene can include one or more transcriptional regulatory sequences and any other nucleic acid, such as introns, that may be necessary for optimal expression of a selected nucleic acid.

“Transgenic animal” means any animal, preferably a non-human mammal, bird or an amphibian, in which one or more of the cells of the animal contain heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA. In the typical transgenic animals described herein, the transgene causes cells to express are reporter gene operably linked to a TSLP promoter or portion thereof.

“Transcription” means the first step in gene expression wherein the message encoded in DNA is transcribed to form RNA that can be used as a template to make proteins.

“Transcription factor” means any of various proteins that interact with DNA and modulate gene expression by activating or inhibiting transcription of a gene.

“Transfection” means the introduction of a nucleic acid, e.g., via an expression vector, into a recipient cell by nucleic acid-mediated gene transfer.

“Treating” means preventing, curing, or ameliorating at least one symptom of the condition or disease.

“Vector” means a nucleic acid molecule capable of transporting another nucleic acid to which it has been linked. A preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors.” In general, expression vectors of utility in recombinant DNA techniques are often in the form of “plasmids” that refer generally to circular double stranded DNA loops that, in their vector form are not bound to the chromosome. In the present specification, “plasmid” and “vector” are used interchangeably as the plasmid is the most commonly used form of vector. However, the invention is intended to include such other forms of expression vectors that serve equivalent functions and that become known in the art subsequently hereto.

### THE INVENTION

The present invention is based on the identification and characterization of the promoter region of the human Thymic Stromal Lymphopoietin (TSLP) gene. The promoter region comprises 3026 base pairs upstream of the putative TSLP transcription start site. The nucleic acid sequence of the TSLP promoter is shown in SEQ ID NO:1. A number of putative transcription factor binding sites within this region have been identified. These sites include, but are not limited to, p53, Activator Protein-1 (AP-1), AP-4, CRF-binding protein 1/c-Jun heterodimer, CAAT/enhancer binding protein α (C/EBP-α), C/EBP-β, GATA-binding factor 1 (GATA-1), GATA-2, GATA-3, GATA-X, Nuclear Factor of Activated T-cells (NFAT), Nuclear Factor κ B (NFκB), Octomer factor 1 (Oct-1), Stimulating protein 1 (Sp-1) and Stress response element (STRE).

In one aspect, the invention comprises isolated TSLP promoter nucleic acid molecules. In one embodiment, the TSLP promoter has the nucleic acid sequence set forth in SEQ ID NO:1. In another embodiment, the TSLP promoter comprises functional portions of the nucleic acid set forth in SEQ ID NO:1 and may include individual or combinations of these portions, particularly those described in Table 1. In a preferred embodiment, the TSLP promoter is operably linked to a reporter gene.

In another aspect, the invention comprises a nucleic acid that is at least about 70%, 80%, 85%, 90% or 95% homologous to the TSLP promoter shown in SEQ ID NO:1 or functional portions thereof or to the complement of the nucleic acid shown as SEQ ID NO:1. Preferred nucleic acids of the invention are vertebrate, preferably mammalian, even more preferably human nucleic acids.

In another aspect, the invention comprises a nucleic acid molecule produced by linking two or more functional portions of the TSLP promoter having the nucleic acid sequence set forth in SEQ ID NO:1. The portions can be the same portions, i.e., multiple repeats, of a combination of different portions. Preferably, the molecule is a construct having TSLP promoter activity comprising at least two identical functional portions of the isolated TSLP promoter.

In another aspect, the invention comprises methods for identifying compounds that function as TSLP promoter-mediated transcription antagonists. In one embodiment, the method comprises contacting a preparation comprising a reporter gene operably linked to a TSLP promoter, preferably cell transfected with a reporter gene operably linked to a TSLP promoter, with a compound and comparing the level of expression of the reporter gene in the absence of the compound with the level of expression in the presence of the compound, wherein a decrease in the level of expression in the presence of the compound relative to the level in the absence of the compound indicates that the compound is a TSLP antagonist. Appropriate compounds can be small molecules, proteins, glycoproteins, nucleic acids, carbohydrates, and lipids.

In a preferred embodiment, the method is performed in the presence (either individually or in combination) of a known activator of TSLP transcription (e.g., a protein kinase C stimulator, such as phorbol-12-myristate-13-acetate (PMA) or a calcium ionophore, such as A23187).

TSLP promoter antagonists identified using the disclosed methods are useful agents or drugs for treating allergic conditions, such as allergic rhinitis, hay fever, perennial rhinitis, seasonal/perennial allergic conjunctivitis, vernal keratoconjunctivitis, giant papillary conjunctivitis, perennial allergic conjunctivitis and atopic keratoconjunctivitis, atopic dermatitis, and allergic (extrinsic) asthma, food reactions, systemic anaphylaxis, allergic pulmonary disease, anaphylaxis, urticaria and angioedema (hives; giant urticaria; angioneurotic edema), hereditary angioedema, mastocytosis, physical allergy to physical stimuli, e.g., cold, sunlight, heat; mild trauma, contact dermatitis, hypersensitivity pneumonitis, allograft rejection, granulomas due to intracellular organisms, drug sensitivity, thyroiditis, encephalomyclitis after rabies vaccination, cryoglobulinemia, cryoglobulinemic glomerulonephritis, histiocytic lymphomas, Severe Combined Immunodeficiency (SCID), and tonsillitis.

In another aspect, the invention comprises methods for identifying compounds that function as TSLP promoter-mediated transcription agonists. In one embodiment, the method comprises contacting a preparation comprising a reporter gene operably linked to an isolated TSLP promoter, preferably a cell transfected with a reporter gene operably linked to a TSLP promoter, with a compound and comparing the level of expression of the reporter gene in the presence of the compound with the level in the absence of the compound, wherein an increase in the level of expression in the presence of the compound relative to level of expression in the absence of the compound indicates that the compound is a TSLP agonist. Appropriate compounds can be small molecules, proteins, glycoproteins, nucleic acids, carbohydrates, and lipids.

In a preferred embodiment, preparation is a homogenate, cell, tissue, or organism. Preferably, the cell is eukaryotic and can be, for example, in a cell culture, tissue culture, or in an animal, Particularly preferred cells are mammalian, including, but not limited to, human, mouse, rat, goat, pig, and chicken cells.

TSLP promoter agonists identified using the disclosed methods are useful as agents or drugs for treating autoimmune diseases, such as Active Chronic Hepatitis, Addison's Disease, Anti-phospholipid Syndrome, Atopic Allergy, Autoimmune Atrophic Gastritis, Achlorhydra Autoimmune, Celiac Disease, Crohn's Disease, Cushing's Syndrome, Dermatomyositis, Diabetes (type I), Discoid Lupus, Erythematosis, Goodpasture's Syndrome, Grave's Disease, Hashimoto's Thyroiditis, Idiopathic Adrenal Atrophy, Idiopathic Thrombocytopenia, Insulin-dependent Diabetes, Lambert-Eaton Syndrome, Lupoid Hepatitis, some cases of Lymphopenia, Mixed Connective Tissue Disease, Multiple Sclerosis, Pemphigoid, Pemphigus Vulgaris, Pernicious Anema, Phacogenic Uveitis, Polyarteritis Nodosa, Polyglandular Auto. Syndromes, Primary Biliary Cirrhosis, Primary Sclerosing Cholangitis, Psoriasis, Raynaud's Syndrome, Reiter's Syndrome, Relapsing Polychondritis, Rheumatoid Arthritis, Schmidt's Syndrome, Limited Scleroderma (or CREST Syndrome), Severe Combined Immunodeficiency Syndrome (SCID), Sjogren's Syndrome, Sympathetic Ophthalmia, Systemic Lupus Erythematosis, Takayasu's Arteritis, Temporal Arteritis, Thyrotoxicosis, Type B Insulin Resistance, Ulcerative Colitis, and Wegener's Granulomatosis.

In one aspect, the present invention is a method of treating allergic conditions or diseases. The method comprises administering an allergic condition treating amount of a TSLP antagonist to an animal susceptible to or suffering from an allergic condition or disease.

In another aspect, the present invention is a method of treating autoimmune diseases. The method comprises administering an autoimmune diseases treating amount of a TSLP agonist to an animal susceptible to or suffering from an autoimmune diseases.

Other features and advantages of the invention will be apparent from the following detailed description and claims.

### TSLP Promoter Nucleic Acids

The human TSLP promoter, shown as SEQ ID NO:1, is 3026 bases in length and contains a number of DNA interaction sites for transcription factors. 140 motifs were identified in human the TSLP promoter set forth in SEQ ID NO:1. Certain motifs are shown in Table 1.

TABLE 1

| Motif | | |
|---|---|---|
| SEQ ID NO: | Sequence | Bases of SEQ ID NO:1 |
| Activator Protein 1 (AP-1) | GTGAATCAG | 1522 to 1530 |
| SEQ ID NO:3 | | |
| AP-1 | CCTGACTCACT | 1989 to 1999 |
| SEQ ID NO:4 | | |
| AP-1 | CTGACTCAC | 1990 to 1998 |
| SEQ ID NO:5 | | |
| AP-4 | GTCAGCGGTG | 1340 to 1349 |
| SEQ ID NO:6 | | |
| CRE-binding protein 1/c-Jun | ATGTTAAGTAATCT | 2889 to 2905 |
| heterodimer | | |
| SEQ ID NO:7 | | |
| CCAAT/enhancer binding | GTGTTTAGCAATGT | 945 to 932 |
| protein α (C/EBP-α) | | |
| SEQ ID NO:8 | | |
| CCAAT/enhancer binding | ATGTTAAGTAATCT | 534 to 547 |
| protein β (C/EBP-β) | | |
| SEQ ID NO:9 | | |
| C/EBP-β | GTGTTTAGCAATGT | 932 to 945 |
| SEQ ID NO:10 | | |
| GATA-binding factor 1 | AGAGATAAGG | 506 to 515 |
| (GATA-1) | | |
| SEQ ID NO:11 | | |
| GATA-1 | TTACAGATAAGGAA | 898 to 911 |
| SEQ ID NO:12 | | |
| GATA-1 | TACAGATAAGGAA | 899 to 911 |
| SEQ ID NO:13 | | |
| GATA-1 | ACAGATAAGG | 900 to 909 |
| SEQ ID NO:14 | | |
| GATA-1 | CCCCATCAGC | 1140 to 1149 |
| SEQ ID NO:15 | | |
| GATA-1 | GGTGATGGGG | 1422 to 1431 |
| SEQ ID NO:16 | | |
| GATA-1 | CCTGATCGGT | 1895 to 1904 |
| SEQ ID NO:17 | | |
| GATA-1 | CGCCATCTCG | 2062 to 2071 |
| SEQ ID NO:18 | | |
| GATA-2 | AGAGATAAGG | 506 to 515 |
| SEQ ID NO:19 | | |
| GATA-2 | ACAGATAAGG | 900 to 909 |
| SEQ ID NO:20 | | |
| GATA-2 | AGCTATCCCA | 1281 to 1290 |
| SEQ ID NO:21 | | |
| GATA-2 | CGCCATCTCG | 2062 to 2071 |
| SEQ ID NO:22 | | |
| GATA-2 | TCTTATCGTT | 2499 to 2508 |
| SEQ ID NO:23 | | |
| GATA-3 | AGAGATAAGG | 506 to 515 |
| SEQ ID NO:24 | | |
| GATA-3 | GAGATAAGG | 507 to 515 |
| SEQ ID NO:25 | | |
| GATA-3 | CAGATTGGG | 676 to 684 |
| SEQ ID NO:26 | | |
| GATA-3 | CAGATAAGG | 901 to 909 |
| SEQ ID NO:27 | | |
| GATA-3 | ATAGATCATA | 1021 to 1030 |
| SEQ ID NO:28 | | |
| GATA-3 | TCAGATCTTT | 2472 to 2481 |
| SEQ ID NO:29 | | |
| GATA-X | AGATAAGGAAA | 902 to 912 |
| SEQ ID NO:30 | | |
| Nuclear Factor of Activated | CAATGGAAAAGA | 33 to 44 |
| T-cells (NFAT) | | |
| SEQ ID NO:31 | | |
| NF-AT | CCTAGGAAAATG | 744 to 755 |
| SEQ ID NO:32 | | |
| NF-AT | TTTTTTCCTTTC | 2935 to 2946 |
| SEQ ID NO:33 | | |
| Nuclear Factor κ B (NFκB) | GGAACTTCCC | 1717 to 1726 |
| SEQ ID NO:34 | | |
| NFκB | GGAAATGCCC | 2780 to 2789 |
| SEQ ID NO:35 | | |
| NFκB | GGGAAATTCC | 2876 to 2885 |
| SEQ ID NO:36 | | |
| NFκB | GGAAATTCCT | 2877 to 2886 |
| SEQ ID NO:37 | | |
| Octomer factor 1 (Oct-1) | AGGATAATGAGGT | 589 to 601 |
| SEQ ID NO:38 | | |
| Oct-1 | ACATAATTACAGA | 892 to 904 |
| SEQ ID NO:39 | | |
| Stimulating protein 1 (Sp-1) | GGGGGGCGGGGGT | 1764 to 1776 |
| SEQ ID NO:40 | | |
| Sp-1 | GGGGCGGGGG | 1766 to 1775 |
| SEQ ID NO:41 | | |
| Stress response element | GCAGGGGG | 1874 to 1881 |
| (STRE) | | |
| SEQ ID NO:42 | | |
| STRE | GCCCCTAA | 2379 to 2386 |
| SEQ ID NO:43 | | |
| STRE | GCCCCTAA | 2512 to 2519 |
| SEQ ID NO:44 | | |
| p53 | AGGCATGTCA | 1223 to 1232 |
| SEQ ID NO:45 | | |

Accordingly, particular embodiments of the invention include the human TSLP promoter provided as SEQ ID NO:1 and functional portions that have TSLP promoter activity, including the motifs given in Table 1. For instance, the TSLP promoter may comprise bases that are at least 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000 or 3050 upstream of the transcriptional start site.

Preferred nucleic acids of the invention comprise a nucleic acid sequence that is at least about 70%, 75%, 80%, 85%, 90%, 95% or 98% homologous to a nucleic acid sequence of SEQ ID NO:1. Even more preferred nucleic acids have a nucleotide sequence that is at least about 99% identical to the nucleotide sequence set forth in SEQ ID NO:1.

Functional portions of the TSLP promoter may be identified by operably linking fragments of the TSLP promoter to the TSLP gene or to a reporter gene and determining the expression of the TSLP gene or the reporter gene. The expression of the TSLP gene or the reporter gene demonstrates that the portion or fragment of the TSLP promoter is functional and has promoter activity.

The nucleic acids of the present invention can be linked to a reporter gene where the expressed reporter gene serves as a marker of transcription at the TSLP promoter.

### Vectors Containing the TSLP Promoter

The invention further provides plasmids and vectors containing the TSLP promoter or portions thereof, which can be used to express a reporter gene in vitro or in a host cell. The host cell may be any prokaryotic or eukaryotic cell. Ligating the polynucleotide sequence into a gene construct, such as an expression vector, and transforming or transfecting into hosts, either eukaryotic (yeast, avian, insect or mammalian) or prokaryotic (bacterial) cells, are standard procedures well known in the art.

Expression vectors contain a nucleic acid encoding a reporter gene, operably linked to a promoter. In one embodiment, the expression vector includes the reporter gene luciferase operably linked to the TSLP promoter of SEQ ID NO:1.

Suitable vectors for the expression of a reporter gene operably linked to the TSLP promoter or a portion thereof include, but are not limited to, pBR322-derived plasmids, pEMBL-derived plasmids, pEX-derived plasmids, pBTac-derived plasmids and pUC-derived plasmids for expression in prokaryotic cells, such as E. coli.

A number of vectors exist for the expression of recombinant proteins in yeast. For instance, YEP24, YIP5, YEP51, YEP52, pYES2, and YRP17 are cloning and expression vehicles useful in the introduction of genetic constructs into S. cerevisiae (see, for example, Broach et al. (1983) in Experimental Manipulation of Gene Expression, ed. M. Inouye Academic Press, p. 83, incorporated by reference herein). These vectors can replicate in E. coli due the presence of the pBR322 ori, and in S. cerevisiae due to the replication determinant of the yeast 2 micron plasmid. In addition, drug resistance markers such as ampicillin can be used.

The preferred mammalian expression vectors contain both prokaryotic sequences, to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papillomavirus (BPV-1), or Epstein-Barr virus (pHEBo, pREP-derived and p205) can be used for transient expression of a reporter gene in eukaryotic cells. The various methods employed in the preparation of the plasmids and transformation of host organisms are well known in the art. For other suitable expression systems for both prokaryotic and eukaryotic cells, as well as general recombinant procedures, see Molecular Cloning a Laboratory Manual, 2nd Ed., ed. by Sambrook, Fritsch and Maniatis (Cold Spring Harbor Laboratory Press: 1989) Chapters 16 and 17.

In some instances, it may be desirable to express the reporter gene by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

In addition to viral transfer methods, non-viral methods can also be employed to cause expression of a reporter gene that is operably linked to the TSLP promoter or a portion thereof in the tissue of an animal. Most non-viral methods of gene transfer rely on normal mechanisms used by mammalian cells for the uptake and intracellular transport of macromolecules. In preferred embodiments, non-viral targeting means of the present invention rely on endocytic pathways for the uptake of the reporter gene operably linked to the TSLP promoter or a portion thereof by the targeted cell. Exemplary targeting means of this type include, for example, liposomal derived systems, poly-lysine conjugates, and artificial viral envelopes.

In other embodiments transgenic animals, described in more detail below could be used to express the reporter gene operably linked to the TSLP promoter or a portion thereof.

### Methods for Identifying Compounds that Modulate TSLP Expression

The invention provides for compounds that modulate (i.e., agonize or antagonize) transcription at the TSLP promoter and treating diseases or conditions caused by, or contributed to by an abnormal TSLP activity, e.g., allergic rhinitis (hay fever), anaphylaxis, asthma, atopic dermatitis cryoglobulinemia, autoimmune diseases, cryoglobulinemic glomerulonephritis, histiocytic lymphomas, rheumatoid arthritis, and tonsillitis. The compounds that can be used for this purpose can be any type of compound, including a protein, a peptide, peptidomimetic, small molecule, lipid, carbohydrate and nucleic acid. A nucleic acid can be, e.g., a gene, an antisense nucleic acid, a ribozyme, or a triplex molecule. A compound of the invention can be a transcriptional activator or inhibitor. Preferred TSLP transcriptional activators or agonists include compounds that are capable of increasing the production of TSLP protein in cells, e.g., compounds capable of upregulating the expression of the TSLP gene. Preferred TSLP transcriptional inhibitors or antagonists include compounds that are capable of decreasing the production of TSLP protein in cells, e.g., compounds capable of downregulating the expression of the TSLP gene.

The invention also provides methods for identifying TSLP transcriptional modulating compounds that are capable of modulating the interaction of the TSLP promoter or a portion thereof and various transcription factors. Transcription factors that interact with various portions of the TSLP promoter include: p53, AP-1, AP-4, CRE-binding protein 1/c-Jun heterodimer, C/EBP-α, C/EBP-β, GATA-1, GATA-2, GATA-3, GATA-X, NFAT, NFκB, Oct-1, Sp-1 and STRE. TSLP transcriptional modulating compounds may bind to the TSLP promoter or portions thereof to promote or inhibit the transcriptional activity at the TSLP promoter. Alternatively, TSLP transcriptional modulating compounds may bind to various transcription factors that interact with the TSLP promoter or portions thereof. In either case, the effect of the TSLP transcriptional modulating compound is to either upregulate or downregulate expression of the TSLP gene.

The compounds of the invention can be identified using various assays depending on the type of compound and activity of the compound that is desired. Set forth below are at least some assays that can be used for identifying TSLP transcriptional modulating compounds. It is within the skill of the art to design additional assays for identifying TSLP transcriptional modulating compounds.

### Cell-Free Assays

Cell-free assays can be used to identify TSLP transcriptional modulating compounds. In a preferred embodiment, cell-free assays for identifying such compounds consist essentially of combining together in a reaction mixture a preparation containing a reporter gene operably linked to a TSLP promoter or portion thereof, a compound or a library of compounds and detecting the expression of the reporter gene. In a preferred embodiment the preparation is a homogenate from tissue having been transformed with a reporter gene operably linked to the TSLP-promoter or portions thereof.

In one embodiment of the invention, a TSLP agonist or TSLP antagonist is a compound that promotes or inhibits, respectively, the interaction of a transcription factor with another molecule, such as a small molecule or a macromolecule. The molecule can be a nucleic acid, such as a transcription factor binding site. The macromolecule can also be a protein.

In many drug screening programs that test libraries of compounds and natural extracts, high throughput assays are desirable to maximize the number of compounds surveyed in a given period of time. Assays that are performed in cell-free systems, such as may be derived with purified or semi-purified proteins, are often preferred as “primary” screens in that they can be generated to permit rapid development and relatively easy detection of an alteration in a molecular target that is mediated by a test compound. Moreover, the effects of cellular toxicity and/or bioavailability of the test compound can be generally ignored in the in vitro system, the assay instead being focused primarily on the effect of the drug on the molecular target as may be manifest in an alteration of binding affinity with upstream or downstream molecules. Accordingly, in an exemplary screening assay of the present invention, the compound of interest is contacted with proteins (i.e., transcription factors) that function upstream (including both activators (enhancers) and repressors of its activity) of TSLP expression. To the mixture of the compound and the transcription factor is then added a composition containing a TSLP promoter or portion thereof. Detection and quantification of complexes of the TSLP promoter with the transcription factor provide a means for determining a compound's efficacy at antagonizing (inhibiting) or agonizing (potentiating) complex formation between a TSLP promoter. The efficacy of the compound can be assessed by generating dose response curves from data obtained using various concentrations of the test compound. Moreover, a control assay can also be performed to provide a baseline for comparison. In the control assay, transcription factor is added to a composition containing TSLP promoter, and the formation of a complex is quantitated in the absence of the test compound.

Complex formation between the transcription factor and the TSLP promoter or portion thereof may be detected by a variety of techniques. Modulation of the formation of complexes can be quantitated using, for example, detectably labeled proteins or nucleic acids, such as radiolabeled, fluorescently labeled, or enzymatically labeled transcription factors TSLP promoters or portions thereof, by immunoassay, or by chromatographic detection.

Typically, it is desirable to immobilize either the transcription factor or the TSLP promoter or portion thereof to facilitate separation of complexes from uncomplexed forms, as well as to accommodate automation of the assay. Binding of the TSLP promoter or portion thereof to the transcription factor, in the presence and absence of a candidate agent, can be accomplished in any vessel suitable for containing the reactants. Examples include microtitre plates, test tubes, and micro-centrifuge tubes. In one embodiment, a fusion protein can be provided that adds a domain that allows the protein to be bound to a matrix. For example, glutathione-S-transferase/ZNFP (GST/ZNFP) fusion proteins can be adsorbed onto glutathione sepharose beads (Sigma Chemical, St. Louis, Mo.) or glutathione derivatized microtitre plates

Other techniques for immobilizing proteins on matrices are also available for use in the subject assay. For instance, a transcription factor and the TSLP promoter or portion thereof can be immobilized utilizing conjugation of biotin and streptavidin. For instance, biotinylated TSLP promoters can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with the transcription factor but which do not interfere with binding to the TSLP promoter or portion thereof can be derivatized to the wells of the plate, and transcription factor trapped in the wells by antibody conjugation. As above, preparations of a transcription factor and a test compound are incubated in the TSLP promoter or portions thereof presenting wells of the plate, and the amount of complex trapped in the well can be quantitated. Exemplary methods for detecting such complexes, in addition to those described above for the GST-immobilized complexes, include immunodetection of complexes using antibodies reactive with the transcription factor and compete with the binding molecule; as well as enzyme-linked assays that rely on detecting an enzymatic activity associated with the binding element, either intrinsic or extrinsic activity. In the instance of the latter, the enzyme can be chemically conjugated or provided as a fusion protein with the transcription factor. To illustrate, the transcription factor can be chemically cross-linked or genetically fused with horseradish peroxidase, and the amount of polypeptide trapped in the complex can be assessed with a chromogenic substrate of the enzyme, e.g. 3,3′-diamino-benzadine terahydrochloride or 4-chloro-1-napthol. Likewise, a fusion protein comprising the polypeptide and glutathione-S-transferase can be provided, and complex formation quantitated by detecting the GST activity using 1-chloro-2,4-dinitrobenzene (Hiabig et al (1974) J Biol Chem 249:7130).

For processes that rely on immunodetection for quantitating the molecule or transcription factor trapped in the complex, antibodies against the transcription factor, can be used. Alternatively, the transcription factor to be detected in the complex can be “epitope tagged” in the form of a fusion protein that includes, in addition to the transcription factor sequence, a second polypeptide for which antibodies are readily available (e.g. from commercial sources). For instance, the GST fusion proteins described above can also be used for quantification of binding using antibodies against the GST moiety. Other useful epitope tags include myc-epitopes (e.g., see Ellison et al. (1991) J Biol Chem 266:21150-21157) that includes a 10-residue sequence from c-myc, as well as the pFLAG system (international Biotechnologies, Inc.) or the pEZZ-protein A system (Pharamacia, N.J.).

Assays for screening drugs that disrupt the interaction of a transcription factor with a nucleic acid can also be performed using, e.g., transcription factor-DNA binding assays, such as those described in U.S. Pat. No. 5,563,036, which is owned by Tularik and is specifically incorporated by reference herein. Other assays for monitoring interaction of a transcription factor to DNA are within the skill in the art and include, e.g., gel shift assays, also referred to herein as “EMSA”. According to this assay, a purified transcription factor protein, or a cellular or nuclear extract prepared from a cell expressing the transcription factor gene are incubated in the presence of a nucleic acid comprising a TSLP promoter or portion thereof. Compounds, e.g., competing nucleic acids (for example, yeast tRNA) can be added to reduce or eliminate non specific binding of transcription factor to the TSLP promoter or portion thereof. After incubation for an adequate amount of time, e.g., 20 minutes, the mixture of nucleic acid and protein is then subjected to gel electrophoresis allowing for the separation of complexes between nucleic acid and proteins and noncomplexed nucleic acids and proteins. In a preferred embodiment, the nucleic acid is radioactively labeled and the protein-DNA complex is detected by autoradiography of the gel. Compounds that modulate the interaction between a transcription factor and a TSLP promoter, can be identified by performing gel shift assays in the presence of varying amounts of test compounds.

Further, an in vitro transcriptional control assay can be used to detect TSLP agonists or TSLP antagonists that can be used for treatment of diseases caused by or contributed to by an aberrant TSLP activity. For example, an in vitro transcription array can be performed comprising a transcription factor, and a reporter construct comprising a TSLP promoter or portions thereof and a nuclear extract. A test compound can then be added to the transcription reaction and transcription of the reporter gene is determined according to methods known in the art.

### Cell-Based Assays

In addition to cell-free assays, such as described above, having identified the TSLP promoter and portions thereof also facilitates the generation of cell-based assays for identifying TSLP transcriptional modulating compounds. Accordingly, in one embodiment, a cell that is capable of expressing a reporter gene operably linked to a TSLP promoter or a portion thereof is incubated with a test compound and the amount of report gene or reporter gene activity produced by the cell is measured and compared to that produced from a cell that has not been contacted with the test compound. The specificity of the compound vis-à-vis modulating TSLP transcriptional activity can be confirmed by various control analysis, e.g., measuring the expression of one or more control genes. Compounds that can be tested include small molecules, proteins, and nucleic acids.

For example, cells in an in vitro culture or organotypic cultures can be engineered to express a reporter gene operably linked to a TSLP promoter or portion thereof. Any of the reporter genes known in the art can be used and may include but are not limited to luciferase or chloramphenicol acetyl transferase. Cells can then be contacted with test compounds. Agonists of transcriptional activity at the TSLP promoter or portions thereof will cause transcriptional activation of the reporter gene as compared to that seen in control cells in the absence of compound or in the absence of the TSLP promoter-reporter gene construct.

For testing antagonist compounds, cells can be contacted with an agonist before being contacted with test compounds and an inhibition of reporter gene transcription or product can be detected. Alternatively, the cell may express some basal level of the reporter gene such that exposure of the cells to the test compound reduces the basal expression of the reporter gene.

### Transgenic Animals

Transgenic animals include human and non-human animals containing a reporter gene under the control of the TSLP promoter or portions thereof as described above. Such animals can be used, e.g., to identify conditions wherein the expression of a reporter gene is initiated from the TSLP promoter or a portion thereof. Conditions of particular usefulness include those that would result in the expression of TSLP and therefore one could identify compounds that inhibit the transcriptional activity of the TSLP promoter or a portion thereof. Transcriptional activity of the TSLP promoter or portions thereof could be determined by detecting the expression of the reporter gene in situ or from cells or tissue biopsies of the transgenic animal.

The transgenic animals discussed herein may be used to generate cell lines that can be used in the above-described cell based assays. While primary cultures derived from these transgenic animals of the invention may be utilized, the generation of continuous cell lines is preferred. For examples of techniques that may be used to derive a continuous cell line from the transgenic animals, see Small et al., 1985, Mol. Cell. Biol. 5:642-648.

Monitoring the influence of compounds on cells may be applied not only in basic drug screening, but also in clinical trials. In such clinical trials, the expression of a panel of genes may be used as a “read out” of a particular drug's therapeutic effect.

These systems may be used in a variety of applications. For example, the cell-based and animal-based model systems may be used to further characterize the TSLP promoter, in particular their role in diseases associated with an aberrant TSLP activity. Thus, the animal-based and cell-based models may be used to identify drugs, pharmaceuticals, therapies and interventions that may be effective in treating disease.

One aspect of the present invention concerns transgenic animals that are comprised of cells (of that animal) that contain a transgene of the present invention and that preferably (though optionally) express an exogenous reporter gene in one or more cells in the animal. A preferred TSLP promoter-reporter transgene can encode the TSLP promoter set forth in SEQ ID NO:1 or may comprise various portions of the TSLP promoter, including those found in SEQ ID NOs:3-45. In preferred embodiments, the expression of the transgene is restricted to specific subsets of cells, tissues or developmental stages utilizing, for example, cis-acting sequences that control expression in the desired pattern. Moreover, temporal patterns of expression can be provided by, for example, conditional recombination systems or prokaryotic transcriptional regulatory sequences.

The transgenic animals of the present invention all include within a plurality of their cells a transgene of the present invention, which transgene alters the phenotype of the “host cell” with respect to regulation of cell growth, death and/or differentiation. Since it is possible to produce transgenic organisms of the invention utilizing one or more of the transgene constructs described herein, a general description will be given of the production of transgenic organisms by referring generally to exogenous genetic material. This general description can be adapted by those skilled in the art to incorporate specific transgene sequences into organisms utilizing the methods and materials described below.

In an exemplary embodiment, the “transgenic non-human animals” of the invention are produced by introducing transgenes into the germline of the non-human animal. Embryonal target cells at various developmental stages can be used to introduce transgenes. Different methods are used depending on the stage of development of the embryonal target cell. The specific line(s) of any animal used to practice this invention are selected for general good health, good embryo yields, good pronuclear visibility in the embryo, and good reproductive fitness. In addition, the haplotype is a significant factor. For example, when transgenic mice are to be produced, strains such as C57BL/6 or FVB lines are often used (Jackson Laboratory, Bar Harbor, Me.). Preferred strains are those with H-2b, H-2d or H-2q haplotypes such as C57BL/6 or DBA/1. The line(s) used to practice this invention may themselves be transgenics, and/or may be knockouts (i.e., obtained from animals that have one or more genes partially or completely suppressed)

In one embodiment, the transgene construct is introduced into a single stage embryo. The zygote is the best target for micro-injection. In the mouse, the male pronucleus reaches the size of approximately 20 micrometers in diameter that allows reproducible injection of 1-2 pl of DNA solution. The use of zygotes as a target for gene transfer has a major advantage in that in most cases the injected DNA will be incorporated into the host gene before the first cleavage (Brinster et al. (1985) PNAS 82:4438-4442). As a consequence, all cells of the transgenic animal will carry the incorporated transgene. This will in general also be reflected in the efficient transmission of the transgene to offspring of the founder since 50% of the germ cells will harbor the transgene.

Normally, fertilized embryos are incubated in suitable media until the pronuclei appear. At about this time, the nucleotide sequence comprising the transgene is introduced into the female or male pronucleus as described below. In some species such as mice, the male pronucleus is preferred. It is most preferred that the exogenous genetic material be added to the male DNA complement of the zygote before being processed by the ovum nucleus or the zygote female pronucleus. It is thought that the ovum nucleus or female pronucleus release molecules that affect the male DNA complement, perhaps by replacing the protamines of the male DNA with histones, thereby facilitating the combination of the female and male DNA complements to form the diploid zygote.

Thus, it is preferred that the exogenous genetic material be added to the male complement of DNA or any other complement of DNA before being affected by the female pronucleus. For example, the exogenous genetic material is added to the early male pronucleus, as soon as possible after the formation of the male pronucleus, which is when the male and female pronuclei are well separated and both are located close to the cell membrane. Alternatively, the exogenous genetic material could be added to the nucleus of the sperm after it has been induced to undergo decondensation. Sperm containing the exogenous genetic material can then be added to the ovum or the decondensed sperm could be added to the ovum with the transgene constructs being added as soon as possible thereafter.

Introduction of the transgene nucleotide sequence into the embryo may be accomplished by any means known in the art such as, for example, microinjection, electroporation, or lipofection. Following introduction of the transgene nucleotide sequence into the embryo, the embryo may be incubated in vitro for varying amounts of time, or reimplanted into the surrogate host, or both. In vitro incubation to maturity is within the scope of this invention. One common method in to incubate the embryos in vitro for about 1-7 days, depending on the species, and then reimplant them into the surrogate host.

For the purposes of this invention a zygote is essentially the formation of a diploid cell that is capable of developing into a complete organism. Generally, the zygote will be comprised of an egg containing a nucleus formed, either naturally or artificially, by the fusion of two haploid nuclei from a gamete or gametes. Thus, the gamete nuclei must be ones that are naturally compatible, i.e., ones that result in a viable zygote capable of undergoing differentiation and developing into a functioning organism. Generally, a euploid zygote is preferred. If an aneuploid zygote is obtained, then the number of chromosomes should not vary by more than one with respect to the euploid number of the organism from that either gamete originated.

In addition to similar biological considerations, physical ones also govern the amount (e.g., volume) of exogenous genetic material that can be added to the nucleus of the zygote or to the genetic material that forms a part of the zygote nucleus. If no genetic material is removed, then the amount of exogenous genetic material that can be added is limited by the amount that will be absorbed without being physically disruptive. Generally, the volume of exogenous genetic material inserted will not exceed about 10 picoliters. The physical effects of addition must not be so great as to physically destroy the viability of the zygote. The biological limit of the number and variety of DNA sequences will vary depending upon the particular zygote and functions of the exogenous genetic material and will be readily apparent to one skilled in the art, because the genetic material, including the exogenous genetic material, of the resulting zygote must be biologically capable of initiating and maintaining the differentiation and development of the zygote into a functional organism.

The number of copies of the transgene constructs that are added to the zygote is dependent upon the total amount of exogenous genetic material added and will be the amount that enables the genetic transformation to occur. Theoretically, only one copy is required; however, generally, numerous copies are utilized, for example, 1,000-20,000 copies of the transgene construct, to insure that one copy is functional. For the present invention, there will often be an advantage to having more than one functioning copy of each of the inserted exogenous DNA sequences to enhance the phenotypic expression of the exogenous DNA sequences.

Any technique that allows for the addition of the exogenous genetic material into nucleic genetic material can be utilized so long as it is not destructive to the cell, nuclear membrane or other existing cellular or genetic structures. The exogenous genetic material is preferentially inserted into the nucleic genetic material by microinjection. Microinjection of cells and cellular structures is known and is used in the art.

Reimplantation is accomplished using standard methods. Usually, the surrogate host is anesthetized, and the embryos are inserted into the oviduct. The number of embryos implanted into a particular host will vary by species, but will usually be comparable to the number of off spring the species naturally produces.

Transgenic offspring of the surrogate host may be screened for the presence and/or expression of the transgene by any suitable method. Screening is often accomplished by Southern blot or Northern blot analysis, using a probe that is complementary to at least a portion of the transgene. Western blot analysis using an antibody against the protein encoded by the transgene may be employed as an alternative or additional method for screening for the presence of the transgene product. Typically, DNA is prepared from tail tissue and analyzed by Southern analysis or PCR for the transgene. Alternatively, the tissues or cells believed to express the transgene at the highest levels are tested for the presence and expression of the transgene using Southern analysis or PCR, although any tissues or cell types may be used for this analysis.

Alternative or additional methods for evaluating the presence of the transgene include, without limitation, suitable biochemical assays such as enzyme and/or immunological assays, histological stains for particular marker or enzyme activities, flow cytometric analysis, and the like. Analysis of the blood may also be useful to detect the presence of the transgene product in the blood, as well as to evaluate the effect of the transgene on the levels of various types of blood cells and other blood constituents.

Progeny of the transgenic animals may be obtained by mating the transgenic animal with a suitable partner, or by in vitro fertilization of eggs and/or sperm obtained from the transgenic animal. Where mating with a partner is to be performed, the partner may or may not be transgenic and/or a knockout; where it is transgenic, it may contain the same or a different transgene, or both. Alternatively, the partner may be a parental line. Where in vitro fertilization is used, the fertilized embryo may be implanted into a surrogate host or incubated in vitro, or both. Using either method, the progeny may be evaluated for the presence of the transgene using methods described above, or other appropriate methods.

The transgenic animals produced in accordance with the present invention will include exogenous genetic material. As set out above, the exogenous genetic material will, in certain embodiments, be a DNA sequence that results in the production of a reporter gene (either agonistic or antagonistic). Further, in such embodiments, the reporter gene will be attached to a, a TSLP promoter or portions thereof, that preferably allows the expression of the transgene product in a specific type of cell that normally expresses TSLP.

Retroviral infection can also be used to introduce transgene into a non-human animal. The developing non-human embryo can be cultured in vitro to the blastocyst stage. During this time, the blastomeres can be targets for retroviral infection (Jaenich, R. (1976) PNAS 73:1260-1264). Efficient infection of the blastomeres is obtained by enzymatic treatment to remove the zona pellucida (Manipulating the Mouse Embryo, Hogan eds. (Cold Spring Harbor Laboratory Press, Cold Spring Harbor, 1986). The viral vector system used to introduce the transgene is typically a replication-defective retrovirus carrying the transgene (Jahner et al. (1985) PNAS 82:6927-6931; Van der Putten et al. (1985) PNAS 82:6148-6152). Transfection is easily and efficiently obtained by culturing the blastomeres on a monolayer of virus-producing cells (Van der Putten, supra; Stewart et al. (1987) EMBO J. 6:383-388). Alternatively, infection can be performed at a later stage. Virus or virus-producing cells can be injected into the blastocoele (Jahner et al. (1982) Nature 298:623-628). Most of the founders will be mosaic for the transgene since incorporation occurs only in a subset of the cells that formed the transgenic non-human animal. Further, the founder may contain various retroviral insertions of the transgene at different positions in the genome that generally will segregate in the offspring. In addition, it is also possible to introduce transgenes into the germ line by intrauterine retroviral infection of the midgestation embryo (Jahner et al. (1982) supra).

### Diseases Treatable with TSLP Transcriptional Modulators

TSLP is an import factor for the development of B-cells. Thus, TSLP transcriptional modulating compounds that upregulate the expression of TSLP can be used to treat individuals who suffer form a lack of or reduced level of B-cells, e.g., autoimmune diseases.

Alternatively, overproduction of TSLP is associated with adverse allergic reactions. Thus, TSLP transcriptional modulating compounds that downregulate the expression of TSLP can be used to treat individuals who suffer for allergic reactions, e.g., allergic rhinitis (hay fever), anaphylaxis, asthma, atopic dermatitis cryoglobulinemia, cryoglobulinemic glomerulonephritis, histiocytic lymphomas, and tonsillitis.

A number of compounds have been shown to either agonize or antagonize the activity of p53, AP-1, AP-4, CRE-binding protein 1/c-Jun heterodimer, C/EBP-α, C/EBP-β, GATA-1, GATA-2, GATA-3, GATA-X, NFAT, NFκB, Oct-1, Sp-1 and STRE. For instance, a number of compounds have been shown to either stimulate or inhibit the transcriptional activity of these transcription factors in various cell types. Accordingly, these compounds would be likely candidates to identify as TSLP agonist or TSLP antagonists. Exemplary compounds include: microtubule-active drugs (taxol, vinblastine, and nocodazol) that stimulate p53; pifithrin-α that inhibits p53; 12-0-tetradecanoylphorbol-13-acetate that stimulates AP-1 and NFκB; curcuminoids that inhibit AP-1; histone deacetylase (HDAC) inhibitors that inhibit p53 and GATA-1 and immunosuppressive drugs FK506 and cyclosporin A (CsA) that inhibit NF-AT. Accordingly, any one or a combination of these compounds may be useful as TSLP agonists or TSLP antagonists.

### Effective Dose of TSLP Transcriptional Agonists or Antagonists

Toxicity and therapeutic efficacy of such TSLP transcriptional modulating compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic effects are preferred. While compounds that exhibit toxic side effects may be used, care should be taken to design a delivery system that targets such compounds to the site of affected tissue to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

### Formulation and Use of TSLP Promoter Agonists and Antagonists

The dosages of TSLP agonist or TSLP antagonist vary according to the age, size, and character of the particular animal and the disease. Skilled artisans can determine the dosages based upon these factors. The agonist or antagonist can be administered in treatment regimes consistent with the disease, e.g., a single or a few doses over a few days to ameliorate a disease state or periodic doses over an extended time to prevent allergy or asthma.

The nature of the disease or condition would determine the route of administering a TSLP transcriptional modulating compound. For instance, some allergic reactions involve the entire body (a systemic reaction) and in some cases could result in anaphylaxis. In this case a TSLP transcriptional modulating compound would be delivered systemically. Alternatively, the disease could be localized to a particular region wherein a TSLP transcriptional modulating compound would be delivered to the area affected. The agonists and antagonists can be administered to the animal in any acceptable manner including by injection, using an implant, and the like. Injections and implants are preferred because they permit precise control of the timing and dosage levels used for administration. The agonists and antagonists are preferably administered parenterally. As used herein parenteral administration means by intravenous, intramuscular, or intraperitoneal injection, or by subcutaneous implant.

Pharmaceutical compositions for use in accordance with the present invention may be formulated in conventional manner using one or more physiologically acceptable carriers or excipients. Thus, the compounds and their physiologically acceptable salts and solvates may be formulated for administration by, for example, injection, inhalation or insufflation (either through the mouth or the nose) or oral, buccal, parenteral, or rectal administration.

For such therapy, the compounds of the invention can be formulated for a variety of loads of administration, including systemic and topical or localized administration. Techniques and formulations generally may be found in Remmington's Pharmaceutical Sciences, Meade Publishing Co., Easton, Pa. For systemic administration, injection is preferred, including intramuscular, intravenous, intraperitoneal, and subcutaneous. For injection, the compounds of the invention can be formulated in liquid solutions, preferably in physiologically compatible buffers such as Hank's solution or Ringer's solution. In addition, the compounds may be formulated in solid form and redissolved or suspended immediately before use. Lyophilized forms are also included.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the compositions may take the form of tablets or lozenges formulated in conventional manner. For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use. When administered by injection, the agonists and antagonists can be administered to the animal in a injectable formulation containing any biocompatible and agonists and antagonists compatible carrier such as various vehicles, adjuvants, additives, and diluents. Aqueous vehicles such as water having no nonvolatile pyrogens, sterile water, and bacteriostatic water are also suitable to form injectable solutions. In addition to these forms of water, several other aqueous vehicles can be used. These include isotonic injection compositions that can be sterilized such as sodium chloride, Ringer's, dextrose, dextrose and sodium chloride, and lactated Ringer's. Nonaqueous vehicles such as cottonseed oil, sesame oil, or peanut oil and esters such as isopropyl myristate may also be used as solvent systems for the compositions. Additionally, various additives which enhance the stability, sterility, and isotonicity of the composition including antimicrobial preservatives, antioxidants, chelating agents, and buffers can be added. Any vehicle, diluent, or additive used would, however, have to be biocompatible and compatible with the agonists and antagonists according to the present invention.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives in addition, detergents may be used to facilitate permeation. Transmucosal administration may be through nasal sprays or using suppositories. For topical administration, the oligomers of the invention are formulated into ointments, salves, gels, or creams as generally known in the art. A wash solution can be used locally to treat an injury or inflammation to accelerate healing.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

To the extent permitted by law, all publications and patents mentioned herein are hereby incorporated by reference in their entirety as if each individual publication or patent was specifically and individually indicated to be incorporated by reference. In case of conflict, the present application, including any definitions herein, shall control.

The invention now being generally described, it will be more readily understood by reference to the following examples, which are included merely for purposes of illustration of certain aspects and embodiments of the present invention, and are not intended to limit the invention.

### EXAMPLES

This invention can be further illustrated by the following examples of preferred embodiments thereof, although it will be understood that these examples are included merely for purposes of illustration and are not intended to limit the scope of the invention unless otherwise specifically indicated.

### Example 1

### TSLP Promoter Luciferase Construct

The TSLP promoter luciferase construct (TSLP-Luc) contains 2424 bps (SEQ ID NO:2) of TSLP promoter sequence cloned in the multiple cloning site of the luciferase reporter backbone vector, pTA-Luc. The 2424 bps of TSLP promoter includes a TATA box (2365-2368 bp of SEQ ID NO:2) in the 3′ distal region, which was placed adjacent to the starting codon of luciferase reporter gene in TA-Luc. The 3′ end was joined with the 5′ end of firefly luciferase in TA-Luc vector.

pTA-Luc is a member of the Mercury™ product line of signal transduction reporter vectors. This vector is designed for analyzing enhancer sequences by assaying for expression of the firefly luciferase (luc) gene from Photinus pyralis. In addition, pTA-Luc contains the minimal TA promoter, the TATA box from the herpes simplex virus thymidine kinase promoter (PTA). Located downstream of PTA is the luciferase reporter gene. pTA-Luc vector maintains constitutive levels of luciferase in transformed cells. The vector backbone also contains a pUC origin of replication, and an ampicillin resistance gene for propagation and selection in E. coli (http://www.bdbiosciences.com/clontech/techinfo/vectors/vectorsT-Z/pTA-Luc.shtml).

### Example 2

### Measuring Activity at the TSLP Promoter

TSLP-Luc activity was assessed by transient transfection of the TSLP-Luc construct in human mast cell leukemia cell line, HMC-1 (Furitsu T, et al., 1993 J. Clin. Invest. 92:1736-44), or 293T/17 cells (ATCC, Manassas, Va.). HMC-1 cells were cultured in Iscove's Modified Dulbecco's medium (Invitrogen, Cat. No. 12440-053) with 10% bovine fetal calf serum. 293T/17 cells were cultured in DMEM (Invitrogen, Cat. No. 111965-092) containing 1 mM pyruvate, 1×MEM nonessential amino acids (Invitrogen) and 10% bovine fetal calf serum.

For transient transfection, the LipoFectamine 2000 reagent kit (Invitrogen, Carlsbad, Calif.) was used following the manufacturer's protocol. 600 ng of TSLP-Luc plasmid and 5 ng of RL-SChA control plasmid were cotransfected in the HMC-1 or 293T/1.7 cells. The control plasmid, RL-SChA (containing the basic chicken β-actin promoter and Renilla luciferase coding region) was used to normalize the transfection efficiencies.

About 18 hours (an overnight incubation period) after transfection, fresh medium was added to the transfected cells, and eight hours later, PMA (100 ng/ml; Sigma) and A23187 (500 ng/ml; Sigma, St Louis, Mo.) were added in the culture medium. Cells were lysed in Passive Cell Lysis buffer (supplied from the Dual-Luciferase Assay kit) and assayed using Dual-Luciferase Reporter Assay system (Promega, Madison, Wis.).

Exposure of HMC-1 cells to the PKC stimulator PMA and the calcium ionophore A23187 increased luciferase activity thus indicating an increase in transcriptional activity at the TSLP promoter in the TSLP-Luc construct. The results are shown in Table 2. Referring to Table 2, the results show luciferase activity readings of the TSLP-Luc as measured by the transient transfection assay in HMC-1 cells. Transcription from the TSLP promoter was induced by the PKC activator phorbol-12-myristate-13-acetate (PMA; 100 ng/ml) and the calcium ionophore A23187 (500 ng/ml).

TABLE 2
TSLP-Luc Transient Transfection in HMC-1 Cells
and Luciferase Reporter Assay Readings

| Plasmid | |
|---|---|
| TA-Luc | TSLP-Luc |

| | | Standard | | Standard |
|---|---|---|---|---|
| Statistics | Mean | Deviation | Mean | Deviation |
| Control buffer | 0.9 | 0.3 | 0.3 | 0.1 |
| PMA/A23187 | 12.2 | 4.9 | 48.6 | 16.6 |

Likewise, exposure of 293T cells to PMA and A23187 increased luciferase activity thus indicating an increase in transcriptional activity at the TSLP promoter in the TSLP-Luc construct. The results are shown in Table 3. Referring to Table 3, the results show luciferase activity readings of the TSLP-Luc as measured by the transient transfection assay in 293T cells. Transcription from the TSLP promoter was induced by the PKC activator phorbol-12-myristate-13-acetate (PMA; 100 ng/ml) and the calcium ionophore A23187 (500 ng/ml).

TABLE 3
TSLP-Luc Transient Transfection in 293T Cells
and Luciferase Reporter Assay Readings

| Plasmid | |
|---|---|
| TA-Luc | TSLP-Luc |

| | | Standard | | Standard |
|---|---|---|---|---|
| Statistics | Mean | Deviation | Mean | Deviation |
| Control buffer | 3.7 | 0.1 | 3.5 | 0.1 |
| PMA/A23187 | 4.5 | 0.1 | 26.0 | 0.6 |

A calcium ionophore alone was also able to stimulate TSLP promoter activity. TSLP-Luc activity was assessed by transient transfection of the TSLP-Luc construct in a human mast cell line, LAD 2. LAD 2 cells were cultured in Iscove's Modified Dulbecco's medium (Invitrogen, Cat. No. 12440-053) with 10% bovine fetal calf serum.

For transient transfection, the LipoFectamine 2000 reagent kit (Invitrogen, Carlsbad, Calif.) was used following the manufacturer's protocol. 600 ng of TSLP-Luc plasmid and 5 ng of RL-SChA control plasmid were cotransfected in the LAD 2 cells following the manufacturer's instructions. The control plasmid, RL-SChA (containing the basis chicken mactin promoter and Renilla luciferase coding region) was used to normalize the transfection efficiencies. About 18 hours (an overnight incubation period) after transfection, fresh medium was added to the transfected cells, and eight hours later, A23187 (500 ng/ml; Sigma, St Louis, Mo.) were added in the culture medium. Cells were lysed in Passive Cell Lysis buffer (supplied from the Dual-Luciferase Assay kit) and assayed using Dual-Luciferase Reporter Assay system (Promega, Madison, Wis.).

Exposure of LAD 2 cells to the calcium ionophore A23187 increased luciferase activity thus indicating an increase in transcriptional activity at the TSLP promoter in the TSLP-Luc construct. Specifically, after exposure to A23187 LAD 2 cells containing the TSLP-Luc construct displayed 171.0 units of F/R luciferase activity, whereas the control construct, TA-Luc, displayed 29.6 units of F/R luciferase activity.

### Example 3

### Identifying TSLP Expression Agonists

As described in Example 2, TSLP-Luc activity is assessed by transient transfection of the TSLP-Luc construct in human mast cell leukemia cell line, HMC-1 (Furitsu T, et al., 1993 J. Clin. Invest. 92:1736-44), or 293T/17 cells (ATCC, Manassas, Va.). HMC-1 cells are cultured in Isvove's Modified Dulbecco's medium (Invitrogen, Cat. No. 12440-053) with 10% bovine fetal calf serum. 293T/17 cells were cultured in DMEM (Invitrogen, Cat. No. 111965-092) containing 1 mM pyruvate, 1×MEM nonessential amino acids (Invitrogen) and 10% bovine fetal calf serum.

For transient transfection, the LipoFectamine 2000 reagent kit (Invitrogen, Carlsbad, Calif.) is used following the manufacturer's protocol. 600 ng of TSLP-Luc plasmid and 5 ng of RL-SChA control plasmid is cotransfected in the cells indicated above. The control plasmid, RL-SCHA (containing the basis chicken β-actin promoter and Renilla luciferase coding region) was used to normalize the transfection efficiencies.

About 18 hours after transfection, fresh medium is added to the transfected cells, and eight hours later, a compound is added in the culture medium. Cells are lysed in Passive Cell Lysis buffer (supplied from the Dual-Luciferase Assay kit) and the expression of luciferase is measured using Dual-Luciferase Reporter Assay system (Promega, Madison, Wis.).

The compound is identified as an agonist if the compound produces and increase in luciferase expression.

### Example 4

### Identifying TSLP Promoter Activity Antagonists

As described in Example 2, TSLP-Luc activity is assessed by transient transfection of the TSLP-Luc construct in human mast cell leukemia cell line, HMC-1 (Furitsu T, et al., 1993 J. Clin. Invest. 92:1736-44), or 293T/17 cells (ATCC, Manassas, Va.). HMC-1 cells are cultured in Isvove's Modified Dulbecco's medium (Invitrogen, Cat. No. 12440-053) with 10% bovine fetal calf serum. 293T/17 cells were cultured in DMEM (Invitrogen, Cat. No. 111965-092) containing 1 mM pyruvate, 1×MEM nonessential amino acids (Invitrogen) and 10% bovine fetal calf serum.

For transient transfection, the LipoFectamine 2000 reagent kit (Invitrogen, Carlsbad, Calif.) is used following the manufacturer's protocol. Typically, 600 ng of TSLP-Luc plasmid and 5 ng of RL-SChA control plasmid is cotransfected in the cells indicated above. The control plasmid, RL-SChA (containing the basic chicken β-actin promoter and Renilla luciferase coding region) was used to normalize the transfection efficiencies.

About 18 hours after transfection, fresh medium is added to the transfected cells, and eight hours later, a compound is added in the culture medium. Cells are lysed in Passive Cell Lysis buffer (supplied from the Dual-Luciferase Assay kit) and the expression of luciferase is measured using Dual-Luciferase Reporter Assay system (Promega, Madison, Wis.). The compound is identified as a TSLP antagonist if the compound produces and decrease in luciferase expression.

The compound may also be identified as an antagonist if it decreases the expression of luciferase after exposing the cells to a known agonist of the TSLP promoter, such as PMA (100 ng/ml; Sigma) and/or A23187 (500 ng/ml; Sigma, St Louis, Mo.). Alternatively, the compound may be identified as an antagonist if it inhibits the expression of luciferase when the cells are exposed to the compound before the cells are exposed to a known agonist of the TSLP promoter, such as PMA (100 ng/ml; Sigma) or A23187 (500 ng/ml; Sigma, St Louis, Mo.).

In the specification, there have been disclosed typical preferred embodiments of the invention and, although specific terms are employed, they are used in a generic and descriptive sense only and not for purposes of limitation, the scope of the invention being set forth in the following claims. Obviously many modifications and variations of the present invention are possible in light of the above teachings. It is therefore to be understood that within the scope of the appended claims the invention may be practiced otherwise than as specifically described.

We claim:

1. An isolated TSLP promoter comprising the nucleic acid sequence set forth in SEQ ID NO:1 or a functional portion thereof having TSLP promoter activity.

2. The isolated TSLP promoter of claim 1 further comprising a structural gene.

3. The isolated TSLP promoter of claim 1 further comprising an operably linked reporter gene.

4. The isolated TSLP promoter of claim 3 wherein the reporter gene is luciferase.

5. The isolated TSLP promoter of claim 4 comprising the nucleic acid sequence set forth in SEQ ID NO:2 or a functional portion thereof having TSLP promoter activity.

6. The isolated TSLP promoter of claim 1 wherein the TSLP promoter is a human TSLP promoter.

7. A vector comprising the isolated TSLP promoter of claim 1.

8. A host cell comprising the isolated TSLP promoter of claim 1.

9. An isolated mammalian TSLP promoter that is at least 70% identical to SEQ ID NO:1.

10. The isolated mammalian TSLP promoter of claim 9 that is a functional portion of the TSLP promoter having TSLP promoter activity.

11. A method of identifying a TSLP antagonist comprising:
contacting a preparation comprising a reporter gene operably linked to an isolated TSLP promoter with a compound; and
comparing the level of expression of the reporter gene in the presence of the compound to the level of expression of the reporter gene in the absence of the compound, wherein a decrease in the level of expression of the reporter gene in the presence of the compound indicates that the compound is a TSLP antagonist.

12. The method of claim 11 wherein the TSLP promoter comprises SEQ ID NO:1 or a functional portion thereof having TSLP promoter activity.

13. The method of claim 11 wherein the reporter gene is luciferase.

14. The method of claim 11 wherein the preparation is a homogenate, cell, tissue, or organism.

15. The method of claim 11 further comprising contacting the preparation with a known activator of TSLP transcription.

16. The method of claim 15 wherein the known activator of TSLP transcription is a compound that activates protein kinase C.

17. The method of claim 16 wherein the compound that activates protein kinase C is PMA.

18. The method of claim 17 wherein the known activator of TSLP transcription is a calcium ionophore.

19. The method of claim 18 wherein the calcium ionophore is A23187.

20. The method of claim 11 wherein the compound is a small molecule, protein, glycoprotein, nucleic acid, lipid, or carbohydrate.

21. A method of identifying a TSLP agonist comprising:
contacting a preparation comprising a reporter gene operably linked to an isolated TSLP promoter with a compound; and
comparing the level of expression of the reporter gene in the presence of the compound to the level of expression of the reporter gene in the absence of the compound, wherein an increase in the level of expression of the reporter gene in the presence of the compound indicates that the compound is a TSLP agonist.

22. The method of claim 21 wherein the TSLP promoter comprises SEQ ID NO:1 or a functional portion thereof having TSLP promoter activity.

23. The method of claim 21 wherein the reporter gene is luciferase.

24. The method of claim 21 wherein the preparation is a homogenate, cell, tissue, or organism.

25. The method of claim 21 wherein the compound is a small molecule, protein, glycoprotein, nucleic acid, lipid, or carbohydrate.

26. A method of treating allergic conditions comprising administering an allergic condition treating amount of a TSLP antagonist to an animal susceptible to or suffering from an allergic condition or disease.

27. The method of claim 26 wherein the TSLP antagonist is a compound that inhibits the activity of transcription factors.

28. The method of claim 26 wherein the allergic conditions are selected from the group consisting of allergic rhinitis, hay fever, perennial rhinitis, seasonal/perennial allergic conjunctivitis, vernal keratoconjunctivitis, giant papillary conjunctivitis, perennial allergic conjunctivitis and atopic keratoconjunctivitis, atopic dermatitis, and allergic (extrinsic) asthma, food reactions, systemic anaphylaxis, allergic pulmonary disease, anaphylaxis, urticaria and angioedema (hives; giant urticaria; angioneurotic edema), hereditary angioedema, mastocytosis, physical allergy to physical stimuli, e.g., cold, sunlight, heat, mild trauma, contact dermatitis, hypersensitivity pneumonitis, allograft rejection, granulomas due to intracellular organisms, some forms of drug sensitivity, thyroiditis, encephalomyelitis after rabies vaccination, cryoglobulinemia, cryoglobulinemic glomerulonephritis, histiocytic lymphomas, Severe Combined Immunodeficiency (SCID), and tonsillitis.

29. A method of treating autoimmune diseases comprising administering an autoimmune diseases treating amount of a TSLP agonist to an animal susceptible to or suffering from an autoimmune diseases.

30. The method of claim 29 wherein the TSLP agonist is a compound that stimulates the activity of transcription factors.

31. The method of claim 29 wherein the autoimmune diseases are selected from the group consisting of: Active Chronic Hepatitis, Addison's Disease, Anti-phospholipid Syndrome, Atopic Allergy, Autoimmune Atrophic Gastritis, Achlorhydra Autoimmune, Celiac Disease, Crohn's Disease, Cushing's Syndrome, Dermatomyositis, Diabetes (type 1), Discoid Lupus, Erythematosis, Goodpasture's Syndrome, Grave's Disease, Hashimoto's Thyroiditis, Idiopathic Adrenal Atrophy, Idiopathic Thrombocytopenia, Insulin-dependent Diabetes, Lambert-Eaton Syndrome, Lupoid Hepatitis, some cases of Lymphopenia, Mixed Connective Tissue Disease, Multiple Sclerosis, Pemphigoid, Pemphigus Vulgaris, Pernicious Anema, Phacogenic Uveitis, Polyarteritis Nodosa, Polyglandular Auto. Syndromes, Primary Biliary Cirrhosis, Primary Sclerosing Cholangitis, Psoriasis, Raynaud's Syndrome, Reiter's Syndrome, Relapsing Polychondritis, Rheumatoid Arthritis, Schmidt's Syndrome, Limited Scleroderma (or CREST Syndrome), Severe Combined Immunodeficiency Syndrome (SCID), Sjogren's Syndrome, Sympathetic Ophthalmia, Systemic Lupus Erythematosis, Takayasu's Arteritis, Temporal Arteritis, Thyrotoxicosis, Type B Insulin Resistance, Ulcerative Colitis, and Wegener's Granulomatosis.

32. A nonhuman transgenic animal comprising an isolated TSLP promoter having the nucleic acid sequence set forth in SEQ ID NO:1 or a functional portion thereof having TSLP promoter activity.

33. A nucleic acid molecule having TSLP promoter activity comprising at least two functional portions of the isolated TSLP promoter having the nucleic acid sequence set forth in SEQ ID NO:1.

34. The nucleic acid construct of claim 33 wherein the functional portions of the TSLP promoter are identical.


SEQUENCE LISTING (45 sequences)

SEQ ID NO: 1 - DNA, 3026 bp, Homo sapiens
tagaaaaagaagtatttatagagaggttctttcaatggaaaagaatttgcttcctgacaatgatcttcatagaaggttaatatgcttaagcacattgagaattcattgaatatctattacatgtagtacagaagacagccctattgtggttatgatgatgaccataagcttaagaaaaactggctttcatgatgaatttgtgtgtggtagagcttgtcatagtctaccccttgtagtggcagtttagaagctcaaaactcacttttgagatcaattgttataattctccagtttttagtaatgcttttctatagccttccctgcacctgattctaaccgccctattcctttgctccattctcttgtctcaccttgactgtttttttaccttttgacaccaatgtatttattgcaaatcacttcaaatgctactggaaaggtaaatagtgaacaaatgaactgctataaaacattacctacttttaaagaactcgcaatctaatagagagataaggttgaaaaacattattgaaatgttaagtaatctatagaagccatagaaagaagtccaaaatagatacggcttacaggataatgaggtcacatcctggtaggagaccacttttggagacatcaaataagatgaacatggcaagacatgaaaatctaacatctcagattgggaagaaaacagctcagagcatgtttagagcatattaccttcagatactgtttgctggaagcctaggaaaatgaaggaaatagggctggaaagacagcttggagccaacgcaggagggataattgtaatctcaaaacaaccttgagtaggttgttttgccataactttttgagcagacaagtaatataatccaggtgatctaagaggtcacataattacagataaggaaattgggggcaaaaggaacaagtgtttagcaatgttttaacagacaaagagacacctgggatgccttagaaagagataccctgcctacctcatctgaacaaagactctacatagatcatagacacaaggggtttttaaaagaggaaacaaaaaaagggaggcagcctggttatacctccagaagaatgcaagtcgaagctcagtgtaggctgctttctgttgttcttgcccccatcagcaatcaagagatgaagccgaaggttctagtgggcactcacactctctggccctacagcaagccactagaggggaaggcatgtcaggtgagggtgacagaagcagccaccatgccctttcctttgtctcttggagctatcccagcccagtgaagaaatctaagtttagctcctgtctgcctatataataatgtccagctgccgaccagaataatctctgatgtcacaatagattaatcaggttttggcatcatcccacaataccctgagaagttggtgatgggggaagagtggacttttttttcaaagaatgctgtggtgactcgagtacaacacatgaagaattccagaaaagagccatttttgaagtcagcggtgaatcagaggttttctgaccctctcgagtgccttctatgaagaagagttcaactctggctccagtttgtcccctactcttttcccgaatccctcgacattccttgctcttttccccaggcaccgcatggcctgtgacatttgctgggactcacacacggcgtccaacctcctttctccgagcgatgactcctaggaacttcccaaggaccagagcgatgcaggctccgacacgcgagggcggggggcgggggtgaggggtgcgatggacgggggagcggaagggaaagagagagagaggggacctgggtgtagagagggcccagggccgcatgagccaagccagggaggagcagggggttcatctcagcaacctgatcggtgtcggcctttccctcccttcaaccctccattggtgttgctggaactgggaagactgctagacagacatcctctagggcactctccctgactcactttgctgtgcttttaagcgctcttttctggatcaacaagagtttgctagtaaaaatctgagcccgccatctcgatgcttctcctgtctctaaaactggtgcccgggctgaggccccaggtgacagacgttttccagtctacgctgcgcggggctaagcctctgcgaggcagagcgcactaaagcgtgcgccgcctccgggagagctgagctcaggacagcatcgtctccgtgcggtcgccgctgggcacccagcagcgcggtctggctccgcttcttgtgtcgctggccttaagagtgggactgtgaaggtggctgggcgctggaatttgtggttttaaaagttcttgacacctgctagaaagtgctagaggactcttcagcccctaaggtgcccctagccaccaagagtaggcgtttttcctgaaattgatatgggaaatttgaaaagtagggtttttgagaacaagccacatcagatctttccccacttatttcaacctcttatcgttcctgcccctaaggttctggtcacggttgcacatttaatgctttcacatggaagtgctgtcgaagactgagactgcaccccaccctccaaagcatcccataagcaagcacagtttcttactagccaaggagaagggaaagcactagttgaaagtggccacaggaaatgctcaaagatgttcagaatgcatgggacatatgcaaggactccaaatccttacagtggctgctggtttcagaaggtgtttctcccaaatagaaaggattggtcacggaaatgccctgtaggagaaagacactggtatccgtttcttaaaggacattcacacttatgaagtgtttagggcaaagcaaaaaggaggaaggtgagggaaattcctgatgactttactgatgttaaaattccatagatttagctactcctttttttttttcctttcacatcatcaattcagaattctataaaggaagagaatgacatggtagaaaatcattggcctaggagaaaagagcccgtagg

SEQ ID NO: 2 - DNA, 2424 bp, Homo sapiens
acatcctggtaggagaccacttttggagacatcaaataagatgaacatggcaagacatgaaaatctaacatctcagattgggaagaaaacagctcagagcatgtttagagcatattaccttcagatactgtttgctggaagcctaggaaaatgaaggaaatagggctggaaagacagcttggagccaacgcaggagggataattgtaatctcaaaacaaccttgagtaggttgttttgccataactttttgagcagacaagtaatataatccaggtgatctaagaggtcacataattacagataaggaaattgggggcaaaaggaacaagtgtttagcaatgttttaacagacaaagagacacctgggatgccttagaaagagataccctgcctacctcatctgaacaaagactctacatagatcatagacacaaggggtttttaaaagaggaaacaaaaaaagggaggcagcctggttatacctccagaagaatgcaagtcgaagctcagtgtaggctgctttctgttgttcttgcccccatcagcaatcaagagatgaagccgaaggttctagtgggcactcacactctctggccctacagcaagccactagaggggaaggcatgtcaggtgagggtgacagaagcagccaccatgccctttcctttgtctcttggagctatcccagcccagtgaagaaatctaagtttagctcctgtctgcctatataataatgtccagctgccgaccagaataatctctgatgtcacaatagattaatcaggttttggcatcatcccacaataccctgagaagttggtgatgggggaagagtggacttttttttcaaagaatgctgtggtgactcgagtacaacacatgaagaattccagaaaagagccatttttgaagtcagcggtgaatcagaggttttctgaccctctcgagtgccttctatgaagaagagttcaactctggctccagtttgtcccctactcttttcccgaatccctcgacattccttgctcttttccccaggcaccgcatggcctgtgacatttgctgggactcacacacggcgtccaacctcctttctccgagcgatgactcctaggaacttcccaaggaccagagcgatgcaggctccgacacgcgagggcggggggcgggggtgaggggtgcgatggacgggggagcggaagggaaagagagagagaggggacctgggtgtagagagggcccagggccgcatgagccaagccagggaggagcagggggttcatctcagcaacctgatcggtgtcggcctttccctcccttcaaccctccattggtgttgctggaactgggaagactgctagacagacatcctctagggcactctccctgactcactttgctgtgcttttaagcgctcttttctggatcaacaagagtttgctagtaaaaatctgagcccgccatctcgatgcttctcctgtctctaaaactggtgcccgggctgaggccccaggtgacagacgttttccagtctacgctgcgcggggctaagcctctgcgaggcagagcgcactaaagcgtgcgccgcctccgggagagctgagctcaggacagcatcgtctccgtgcggtcgccgctgggcacccagcagcgcggtctggctccgcttcttgtgtcgctggccttaagagtgggactgtgaaggtggctgggcgctggaatttgtggttttaaaagttcttgacacctgctagaaagtgctagaggactcttcagcccctaaggtgcccctagccaccaagagtaggcgtttttcctgaaattgatatgggaaatttgaaaagtagggtttttgagaacaagccacatcagatctttccccacttatttcaacctcttatcgttcctgcccctaaggttctggtcacggttgcacatttaatgctttcacatggaagtgctgtcgaagactgagactgcaccccaccctccaaagcatcccataagcaagcacagtttcttactagccaaggagaagggaaagcactagttgaaagtggccacaggaaatgctcaaagatgttcagaatgcatgggacatatgcaaggactccaaatccttacagtggctgctggtttcagaaggtgtttctcccaaatagaaaggattggtcacggaaatgccctgtaggagaaagacactggtatccgtttcttaaaggacattcacacttatgaagtgtttagggcaaagcaaaaaggaggaaggtgagggaaattcctgatgactttactgatgttaaaattccatagatttagctactcctttttttttttcctttcacatcatcaattcagaattctataaaggaagagaatgacatggtagaaaatcattggcctaggagaaaagagcccgtagg

SEQ ID NO: 3 - DNA, 9 bp, Artificial Sequence
  AP-1 MOTIF
gtgaatcag

SEQ ID NO: 4 - DNA, 11 bp, Artificial Sequence
  AP-1 MOTIF
cctgactcact

SEQ ID NO: 5 - DNA, 9 bp, Artificial Sequence
  AP-1 MOTIF
ctgactcac

SEQ ID NO: 6 - DNA, 10 bp, Artificial Sequence
  AP-4 MOTIF
gtcagcggtg

SEQ ID NO: 7 - DNA, 14 bp, Artificial Sequence
  CRE BINDING PROTEIN 1/c-JUN HETERODIMER MOTIF
atgttaagtaatct

SEQ ID NO: 8 - DNA, 14 bp, Artificial Sequence
  C/EBP-ALPHA MOTIF
gtgtttagcaatgt

SEQ ID NO: 9 - DNA, 14 bp, Artificial Sequence
  C/EBP-BETA MOTIF
atgttaagtaatct

SEQ ID NO: 10 - DNA, 14 bp, Artificial Sequence
  C/EPB-BETA MOTIF
gtgtttagcaatgt

SEQ ID NO: 11 - DNA, 10 bp, Artificial Sequence
  GATA-BINDING FACTOR 1 MOTIF
agagataagg

SEQ ID NO: 12 - DNA, 14 bp, Artificial Sequence
  GATA-BINDING FACTOR 1 MOTIF
ttacagataaggaa

SEQ ID NO: 13 - DNA, 13 bp, Artificial Sequence
  GATA-BINDING FACTOR 1 MOTIF
tacagataaggaa

SEQ ID NO: 14 - DNA, 10 bp, Artificial Sequence
  GATA-BINDING FACTOR 1 MOTIF
acagataagg

SEQ ID NO: 15 - DNA, 10 bp, Artificial Sequence
  GATA-BINDING FACTOR 1 MOTIF
ccccatcagc

SEQ ID NO: 16 - DNA, 10 bp, Artificial Sequence
  GATA-BINDING FACTOR 1 MOTIF
ggtgatgggg

SEQ ID NO: 17 - DNA, 10 bp, Artificial Sequence
  GATA-BINDING FACTOR 1 MOTIF
cctgatcggt

SEQ ID NO: 18 - DNA, 10 bp, Artificial Sequence
  GATA-BINDING FACTOR 1 MOTIF
cgccatctcg

SEQ ID NO: 19 - DNA, 10 bp, Artificial Sequence
  GATA-BINDING FACTOR 2 MOTIF
agagataagg

SEQ ID NO: 20 - DNA, 10 bp, Artificial Sequence
  GATA-BINDING FACTOR 2 MOTIF
acagataagg

SEQ ID NO: 21 - DNA, 10 bp, Artificial Sequence
  GATA-BINDING FACTOR 2 MOTIF
agctatccca

SEQ ID NO: 22 - DNA, 10 bp, Artificial Sequence
  GATA-BINDING FACTOR 2 MOTIF
cgccatctcg

SEQ ID NO: 23 - DNA, 10 bp, Artificial Sequence
  GATA-BINDING FACTOR 2 MOTIF
tcttatcgtt

SEQ ID NO: 24 - DNA, 10 bp, Artificial Sequence
  GATA-BINDING FACTOR 3 MOTIF
agagataagg

SEQ ID NO: 25 - DNA, 9 bp, Artificial Sequence
  GATA-BINDING FACTOR 3 MOTIF
gagataagg

SEQ ID NO: 26 - DNA, 9 bp, Artificial Sequence
  GATA-BINDING FACTOR 3 MOTIF
cagattggg

SEQ ID NO: 27 - DNA, 9 bp, Artificial Sequence
  GATA-BINDING FACTOR 3 MOTIF
cagataagg

SEQ ID NO: 28 - DNA, 10 bp, Artificial Sequence
  GATA-BINDING FACTOR 3 MOTIF
atagatcata

SEQ ID NO: 29 - DNA, 10 bp, Artificial Sequence
  GATA-BINDING FACTOR 3 MOTIF
tcagatcttt

SEQ ID NO: 30 - DNA, 11 bp, Artificial Sequence
  GATA-BINDING FACTOR X MOTIF
agataaggaaa

SEQ ID NO: 31 - DNA, 12 bp, Artificial Sequence
  NFAT MOTIF
caatggaaaaga

SEQ ID NO: 32 - DNA, 12 bp, Artificial Sequence
  NFAT MOTIF
cctaggaaaatg

SEQ ID NO: 33 - DNA, 12 bp, Artificial Sequence
  NFAT MOTIF
ttttttcctttc

SEQ ID NO: 34 - DNA, 10 bp, Artificial Sequence
  NUCLEAR FACTOR KAPPA B MOTIF
ggaacttccc

SEQ ID NO: 35 - DNA, 10 bp, Artificial Sequence
  NUCLEAR FACTOR KAPPA B MOTIF
ggaaatgccc

SEQ ID NO: 36 - DNA, 10 bp, Artificial Sequence
  NUCLEAR FACTOR KAPPA B MOTIF
gggaaattcc

SEQ ID NO: 37 - DNA, 10 bp, Artificial Sequence
  NUCLEAR FACTOR KAPPA B MOTIF
ggaaattcct

SEQ ID NO: 38 - DNA, 13 bp, Artificial Sequence
  OCTOMER FACTOR 1 MOTIF
aggataatgaggt

SEQ ID NO: 39 - DNA, 13 bp, Artificial Sequence
  OCTOMER FACTOR 1 MOTIF
acataattacaga

SEQ ID NO: 40 - DNA, 13 bp, Artificial Sequence
  STIMULATING PROTEIN-1 MOTIF
ggggggcgggggt

SEQ ID NO: 41 - DNA, 10 bp, Artificial Sequence
  STIMULATING PROTEIN-1 MOTIF
ggggcggggg

SEQ ID NO: 42 - DNA, 8 bp, Artificial Sequence
  STRESS RESPONSIVE ELEMENT (STRE)  MOTIF
gcaggggg

SEQ ID NO: 43 - DNA, 8 bp, Artificial Sequence
  STRESS RESPONSIVE ELEMENT (STRE) MOTIF
gcccctaa

SEQ ID NO: 44 - DNA, 8 bp, Artificial Sequence
  STRESS RESPONSIVE ELEMENT (STRE) MOTIF
gcccctaa

SEQ ID NO: 45 - DNA, 10 bp, Artificial Sequence
  P53 BINDING MOTIF
aggcatgtca